(19) United States
(12) Patent Application Publication
(10) Pub. No.: US 2007/0232718 A1
(43) Pub. Date: Oct. 4, 2007

(54) Radiopaque dental materials

(76) Inventors: Weitao Jia, Wallingford, CT (US); Shuhua Jin, Wallingford, CT (US)

Correspondence Address: Ann M. Knab;Pentron Corporation, 53 North Plains Industrial Road, Wallingford, CT, 06492, US

(21) Appl. No.: 11/391,735

(22) Filed: Mar. 28, 2006

(51) Int. Cl. A61K 6/08

(52) U.S. Cl. 523/116; 524/408

(57) ABSTRACT

Highly esthetic dental compositions have both good radiopacity and optical opacity. It has been found that the addition of bismuth fluoride to dental material compositions provides these desirable properties. Bismuth fluoride is relatively inexpensive and easily obtainable.

### FIELD OF THE INVENTION

This invention relates to radiopaque dental materials, such as resins for restorative dentistry, and more particularly to dental composite materials that are useful as crown and bridge materials, space maintainers, tooth replacement appliances, splints, partial crowns, dentures, teeth, jackets, as reconstructive materials, restorative materials, filling materials, inlays, onlays, facings, veneers, facets, connectors, abutments, dowels/posts, dental adhesives, cements, sealants and the like.

### BACKGROUND

Dental resin composite compositions usually possess some radioapacity, which is typically provided in the form of radiopaque fillers. The radiopacity is usually inadequate in comparison to natural dentition, such as enamel. This is due to the use of heavy metal elements, such as Zr, Ba, Ca, Sr, Sb, and others commonly known and suitable for dental applications that are typically combined with an oxide, silicate, phosphate, chloride, fluoride, sulfate and the like. The compounds are both x-ray opaque and very optically opaque, which make the dental compositions less pleasingly esthetic. In order to render the compositions more esthetically acceptable, the amount of heavy metal elements may be reduced, but this compromises the radiopacity and provides insufficient results to the dentist.

U.S. Pat. No. 4,629,746 is directed to dental material compositions having rare earth metal fluorides that provide radiopacity to the dental material. Ytterbium fluoride is used with particular preference. Although the composition provides enhanced radiopacity and good optical esthetics, the pure form of the rare earth materials tends to be difficult to obtain, and can be quite costly.

Commonly assigned U.S. patent application Nos. 20040202985, 20050066854 and 20050069836 are directed to dental root canal filling materials containing a thermoplastic material and a radiopaque agent. The main concern here is that the filling material not be transparent to X-rays so that the dentist is able to see the location of the filling material. There is no requirement that the filling material have good optical opacity, since it is used in the internal area of the tooth structure, not the exterior. There is no need to provide highly esthetically pleasing dental filling materials since they are not viewed externally.

There accordingly remains a need in the art for high quality inexpensive radiopacity agents for use in dental restorative materials. It would be beneficial to provide a radiopaque agent that not only provides optimal radiopacity, but also imparts desirable esthetic qualities to the dental material.

### SUMMARY

These and other objects and advantages are accomplished by the highly esthetic dental compositions of the present invention having both good radiopacity and optical opacity. It has been found that the addition of bismuth fluoride to dental material compositions provides these desirable properties. Bismuth fluoride is relatively inexpensive and easily obtainable.

### DETAILED DESCRIPTION OF PREFERRED EMBODIMENTS

As will be appreciated, the present invention provides dental materials containing a heavy metal fluoride compound, bismuth fluoride (BiF₃). The bismuth fluoride compound enhances the radiopacity without introducing significant optical opacity to the dental material. Additionally, the fluoride in the compound is beneficial in contributing to a decrease in dental caries. The optical opacity, which is the percentage of light-blocking property exhibited by the material, provided by the BiF₃ to the dental material is in the range of about 60 to about 70% when measured with a colorimeter instrument. Colorimeters are optical reading devices that can assess what wavelengths are reflected back to its sensors. Since the optical opacity of dentin is approximately 67 to approximately 70%, BiF₃ provides very natural esthetic qualities to the dental materials.

The bismuth fluoride is preferably added in the form of a powder. The average particle size can vary and is in the range of from about 0.005 to about 10 microns, particularly in the range of about 0.05 to about 5 microns, and most preferably in the range of about 0.1 to about 3 microns.

The bismuth fluoride is a filler and depending upon the end use of the dental material, may be present in the range of about 0.1 to about 50 percent by weight of the total composition, preferably in the range of about 1 to about 30 percent by weight and most preferably in the range of about 5 to about 20 percent by weight of the composition. In the most preferred bismuth fluoride filler range, the optical opacity is in the range of about 50 to 75% and the radiopacity is equivalent to Al metal having a thickness between about 4 and 5 mm.

The filler material can be used to form dental composites and restorations in accordance with known procedures. In addition to the bismuth fluoride agent, the dental material contains a polymeric matrix portion. The polymeric matrix is selected from those known in the art of dental materials, including those listed in commonly assigned U.S. Pat. Nos. 6,013,694 and 6,270,562, 6,730,715, and commonly assigned copending U.S. patent application Nos. 20060009540 and 20050192374, all of which are incorporated by reference herein. The polymeric matrix materials include but are not limited to expandable monomers, liquid crystal monomers, ring-opening monomers of epoxide resins, polyamides, acrylates, polyesters, polyolefins, polymides, polyarylates, polyurethanes, vinyl esters or epoxy-based materials. Other polymeric matrices include styrenes, styrene acrylonitriles, ABS polymers, polysulfones, polyacetals, polycarbonates, polyphenylene sulfides, and the like. These polymeric matrices are derived from curing polymeric matrix precursor compositions. Such precursor compositions are well-known in the art, and may be formulated as one-part, two-part, or other compositions, depending on the components.

Preferred materials include those based on acrylic and methacrylic monomers, for example those disclosed in U.S. Pat. Nos. 3,066,112, 3,179,623, and 3,194,784 to Bowen; U.S. Pat. Nos. 3,751,399 and 3,926,906 to Lee et al.; and commonly assigned U.S. Pat. No. 5,276,068 to Waknine and U.S. Pat. No. 5,969,000, all of which are herein incorporated by reference in their entirety. Especially preferred methacrylate monomers include the condensation product of bisphenol A and glycidyl methacrylate, 2,2′-bis [4-(3-methacryloxy-2-hydroxy propoxy)-phenyl] propane (hereinafter abbreviated BIS-GMA), the condensation product of ethoxylated bisphenol A and glycidyl methacrylate, (hereinafter EBPA-DMA), and the condensation product of 2 parts hydroxymethylmethacrylate and 1 part triethylene glycol bis(chloroformate) (hereinafter PCDMA). Polyurethane dimethacrylates (hereinafter abbreviated to PUDMA) are also commonly-used principal polymers suitable for use in the present invention.

The polymeric matrix precursor composition may further comprise a co-polymerizable diluent monomer. Such monomers are generally used to adjust the viscosity of the polymerizable composition, which affects wettability of the composition. Suitable diluent monomers include, without limitation, hydroxyalkyl methacrylates, such as 2-hydroxyethyl methacrylate, 1,6-hexanediol dimethacrylate, and 2-hydroxypropyl methacrylate; glyceryl dimethacrylate; ethyleneglycol methacrylates, including ethyleneglycol methacrylate, diethyleneglycol dimethacrylate, triethyleneglycol dimethacrylate and tetraethyleneglycol dimethacrylate; or diisocyanates, such as 1,6-hexamethylene diisocyanate. Triethyleneglycol dimethacrylate (TEGDMA) is particularly preferred for use in the present invention.

The polymeric matrix precursor composition typically includes polymerization initiators, polymerization accelerators, ultra-violet light absorbers, anti-oxidants, fluorescent whitening agents, and other additives well known in the art. The polymer matrices may be visible light curing, self-curing, dual curing, and vacuum-, heat, and pressure-curable compositions as well as any combination thereof. Visible light curable compositions employ light-sensitive compounds such as benzil diketones, and in particular, dl-camphorquinone in amounts ranging from about 0.05 to 0.5 weight percent. UV absorbers are particularly desirable in the visible light curable compositions in order to avoid discoloration of the resin form any incident ultraviolet light. Suitable UV absorbers are the various benzophenones, particularly UV-9 and UV-5411 available from American Cyanamid Company, and benzotriazoles known in the art, particularly 2-(2′-hydroxy-5′-methylphenyl)benzotriazole, sold under the trademark TINUVIN P by Ciba-Geigy Corporation, Ardsley, N.Y. in amounts ranging from about 0.05 to about 5.0 weight percent.

In the self-curing compositions, a polymerization accelerator may be included in the polymerizable monomer composition. The polymerization accelerators suitable for use include the various organic tertiary amines well known in the art, generally aromatic tertiary amines, such as dimethyl-p-toluidine, dihydroxyethyl-p-toluidine and the like, in amounts ranging from about 0.05 to about 4.0 weight percent, and generally acrylate derivatives such as dimethylaminoethyl methacrylate and particularly, diethylaminoethyl methacrylate in amounts ranging from about 0.05 to 0.5 weight percent.

The heat and pressure curable compositions include, in addition to the monomeric components, a heat cure initiator such as benzoyl peroxide, 1,1′-azobis(cyclohexanecarbonitrile), or other suitable free radical initiators. Particularly suitable free radical initiators are lauroyl peroxide, tributyl hydroperoxide, AIBN and, more particularly benzoyl peroxide or 1,1′-azobis(cyclohexanecarbonitrile).

In addition to the bismuth fluoride agent that is added in the form of a filler, additional filler materials may be present. The total amount of filler is determined by the specific function of the filled materials, being in the range from about 5 to 95% by weight of the total dental material. Fillers in the form of powder and/or fibers may be present in an amount up to about 80% by weight, and preferably about 70% by weight. Suitable fillers include those capable of being covalently bonded to the polymeric matrix itself or to a coupling agent that is covalently bonded to both. Fillers include silica, silicate glass, quartz, barium silicate, strontium silicate, barium borosilicate, strontium borosilicate, borosilicate, lithium silicate, amorphous silica, ammoniated or deammoniated calcium phosphate and alumina, zirconia, tin oxide, and titania, among other conventional fillers such as those disclosed in commonly assigned U.S. Pat. Nos. 4,544,359 and 4,547,531 to Waknine (which are incorporated herein by reference), while possible coupling agents include silanes, zirconates, and titanates. Preferably, the additional filler is barium borosilicate in an amount between about 5% and about 85% by weight of the total composite composition. Examples of glass fillers include those barium borosilicate or other suitable glass fillers commercially available from Schott Electronic Packaging GmbH (Landshut, Germany) under the product codes of GM 27884, G018161, G018-159 or 8235.

The compositions can further comprise other additives, for example pigments; anti-oxidants, for example BHT (2,6-di-tert-butyl-4-methylphenol) or hydroquinone methyl ether in amounts in the range from about 0.1 to about 0.3% by weight of the polymerizable components; ultraviolet stabilizers to prevent discoloration, for example benzophenones such as 2-hydroxy-4-methoxybenzophenone, benzotriazoles, such as 2-(2′-hydroxy-5′-methylphenyl) benzotriazole, 2-(2-hydroxy-5-tert-octylphenyl) benzotriazole (available under the trade name UV-54 from American Cyanamid Company) and other derivatives thereof; fluorescent whitening agents such as 2,5-bis(5-tert-butyl-2-benzoxazole) thiophene (available under the trade name UV-OB); trace amounts of FDA and FDC approved dyes, for example iron oxides, yellow No. 5, yellow No. 6, and the like; and other additives known in the art.

Medicaments can also be included in the compositions in a therapeutically effective amount, for example to relieve pain, prevent infection, prevent inflammation, alleviate sensitivity, and the like. Such amounts are generally about 0.001% to about 10% by weight of the total composition, more commonly about 0.001 % to about 2% by weight of the total composition. Suitable medicaments include but are not limited to pain relieving agents such as Novocaine (procaine hydrochloride), Benzocain (ethyl aminobenzoate), ascorbic acid, butacaine sulfonate, and dibutacaine hydrochloride; antibiotics such as sulfadiazine, procaine penicillin, aureomycin, streptomycin, tetramycin, chloramphenicol, butabarbital, diethyl stilbestrol, and the like; anti-inflammation agents such as p-aminosalicylic acid, aspirin, chlorohexadine, and the like; and desensitizing agents such as sodium fluoride, potassium nitrate, and the like.

The following non-limiting examples illustrate the invention.

### EXAMPLES

A commercially available composite resin, Artiste™ Nano-Hybrid Flowable Composite available from Pentron Clinical Technologies, LLC, Wallingford, Conn. was used to test optical opacity and radiopacity of various radiopaque agents. The radiopaque agents were mixed into the Artiste™ composite in the percentages listed in Table 1 below. After reducing the moisture (removing porosities) from the composite materials, sample disks of 15 mm in diameter and 1.0 mm in thickness were made with a metal mold between two glass slides and visible-light cured for one minute with a Sculpture Plus™ Curing Light available from Pentron Clinical Technologies, LLC, Wallingford, Conn. The cured solid composite sample disks were then removed from the mold and tested for optical opacity by a ColorWalk™ colorimeter instrument, model #2000 available from Seradyn Photovolt Instruments, IN.

The results showing the percentage of optical opacity (percentage of light blocking property) produced by the presence of the radiopaque agents are presented in Table 1 below. The Artiste™ Composite was also tested as a reference, which showed an optical opacity of about 61%. Natural tooth structure of dentin has an optical opacity in the range of about 67-70%.

TABLE 1
Percent Optical Opacity.

| Opacifying | | Artiste ™ | | | |
|---|---|---|---|---|---|
| fillers | | Composite | | | |
| in the | Natural | with no | | | |
| Artiste ™ | Tooth | opacifying | | | |
| Composite | Dentin | agent | BiF₃ | BiOCl | ZrO₂ |
| 33% | | | 84% | | |
| by wt. | | | | | |
| 25% | | | 77% | | |
| 16.7% | | | 71% | | |
| 10% | | | 63% | 87% | 94% |
| 5% | | | 51% | | |
| 0% | 67-70% | 61% | | | |

From the results, one can see that the BiF₃ has a much lower optical opacity when comparing the same level of other common X-ray opacifying fillers like BiOCl and ZrO₂ in a dental resin composition. The addition of BiF₃ to dental materials provides effective radiopacity to the material while imparting good optical opacity to produce an esthetically pleasing appearance very close to the natural look of dentin. Optical opacity above about 70% produces effects that tend to look less natural and less like tooth structure.

Radiopacity is measured according to ISO 4049 for Dentistry—Resin-Based Filling Materials. As shown in Table 2 below, the radiopacity is in the range of 3-5 mm of the equivalent of pure aluminum metal thickness for the opacifying agents. In order to claim that a material is radiopaque, a cured composite sample with two-millimeter thickness should have the X-ray density shown on a dental x-ray film against a pure aluminum (at least 99.5% pure) metal equal to at least the X-ray density of an aluminum metal tab with a two-millimeter thickness. In practice, however, a dentist always prefers a more intensified X-ray image showing a filling material, distinguishing it from a natural radio-opaque tooth structure. Therefore, if a dental restorative material is very radio-opaque on an X-ray picture but at the same time is also esthetically pleasing visually (without the obvious demarcations between the natural tooth structure and the filling material when filled), it is a desirable restorative material.

Table 2 displays the results of X-ray intensity of BiF3, BiOCl and ZrO₂ filler-containing compositions in reference to the equivalence of different aluminum metal thicknesses up to 5 millimeters. If the X-ray intensity of the material is greater than a 5 mm equivalence, a 5+ mm equivalence is reported.

TABLE 2
Measurement of Radiopacity.

| | | | Amount of | Amount of |
|---|---|---|---|---|
| | | Amount of | radiopacity | radiopacity |
| Amount of | | radiopacity | in a | in a |
| opacifying | No | in samples | sample | sample |
| fillers in the | opacifying | containing | containing | containing |
| composite | agent | BiF₃ | BiOCl | ZrO₂ |
| 33% | | Al metal | | |
| by wt. | | 5+ mm | | |
| 25% | | Al metal | | |
| | | 5+ mm | | |
| 16.7% | | Al metal | | |
| | | 5 mm | | |
| 10% | | Al metal | 4+ mm | 3+ mm |
| | | 4+ mm | | |
| 5% | | Al metal | | |
| | | 4 mm | | |
| 0% | Al metal | | | |
| additional | 3 mm | | | |
| opacifying | | | | |
| filler (Artiste | | | | |
| Flow composite | | | | |
| as is) | | | | |

As will be appreciated, the present invention provides a dental material composition have optimal radiopaque properties and good optical opacity. Dental materials formed herein are useful in the formation of highly esthetic dental restorative materials, including, but not limited to, bridges, space maintainers, tooth replacement appliances, splints, crowns, partial crowns, dentures, teeth, jackets, inlays, onlays, facings, veneers, facets, cylinders, abutments, and connectors.

While various descriptions of the present invention are described above, it should be understood that the various features can be used singly or in any combination thereof. Therefore, this invention is not to be limited to only the specifically preferred embodiments depicted herein.

Further, it should be understood that variations and modifications within the spirit and scope of the invention may occur to those skilled in the art to which the invention pertains. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. A dental material comprising:
BiF₃ and
a polymeric matrix precursor composition;
wherein the dental material exhibits a radiopacity equivalent to an aluminum tab having a thickness between about 3 and about 5 mm.

2. The dental material of claim 1 wherein the dental material exhibits a radiopacity equivalent to an aluminum tab having a thickness between about 4 and about 5 mm.

3. The dental material of claim 1 wherein the dental material exhibits a radiopacity equivalent to an aluminum tab having a thickness greater than about 5 mm.

4. The dental material of claim 1 wherein the dental material exhibits an optical opacity in the range of about 60 to about 70% when measured by a colorimeter.

5. The dental material of claim 1 wherein the BiF₃ is present in the range of about 0.1 to about 50 percent by weight of the total composition of the dental material.

6. The dental material of claim 1 wherein the BiF₃ is present in the range of about 1 to about 30 percent by weight of the total composition of the dental material.

7. The dental material of claim 1 wherein the BiF₃ is present in the range of about 5 to about 20 percent by weight of the total composition of the dental material.

8. The dental material of claim 1 wherein the BiF₃ is added in the form of a powder having an average particle size in the range of from about 0.005 to about 10 microns.

9. The dental material of claim 1 wherein the BiF₃ is added in the form of a powder having an average particle size in the range of from about 0.05 to about 5 microns.

10. The dental material of claim 1 wherein the BiF₃ is added in the form of a powder having an average particle size in the range of from about 0.1 to about 3 microns.

11. The dental material of claim 1 wherein the polymeric matrix material comprises expandable monomers, liquid crystal monomers, ring-opening monomers of epoxide resins, polyamides, acrylates, polyesters, polyolefins, polymides, polyarylates, polyurethanes, vinyl esters, epoxy-based materials, styrenes, styrene acrylonitriles, ABS polymers, polysulfones, polyacetals, polycarbonates, polyphenylene sulfides or mixtures thereof.

12. The dental material of claim 11 wherein the acrylates comprise the condensation product of bisphenol A and glycidyl methacrylate, 2,2′-bis [4-(3-methacryloxy-2-hydroxy propoxy)-phenyl] propane (hereinafter abbreviated BIS-GMA), the condensation product of ethoxylated bisphenol A and glycidyl methacrylate, (hereinafter EBPA-DMA), and the condensation product of 2 parts hydroxymethylmethacrylate and 1 part triethylene glycol bis(chloroformate) (hereinafter PCDMA), polyurethane dimethacrylates (hereinafter abbreviated to PUDMA), or mixtures thereof.

13. The dental material of claim 11 wherein the polymeric matrix material further comprises a co-polymerizable diluent monomer.

14. The dental material of claim 13 wherein the co-polymerizable diluent monomer comprises hydroxyalkyl methacrylates, glyceryl dimethacrylate, ethyleneglycol methacrylates, diisocyanates, or mixtures thereof.

15. The dental material of claim 14 wherein the hydroxyalkyl methacrylates comprise 2-hydroxyethyl methacrylate, 1,6-hexanediol dimethacrylate, 2-hydroxypropyl methacrylate, or mixtures thereof.

16. The dental material of claim 14 wherein the ethyleneglycol methacrylates comprise ethyleneglycol methacrylate, diethyleneglycol dimethacrylate, triethyleneglycol dimethacrylate, tetraethyleneglycol dimethacrylate or mixtures thereof.

17. The dental material of claim 14 wherein the diisocyanates comprise 1,6-hexamethylene diisocyanate, triethyleneglycol dimethacrylate (TEGDMA) or mixtures thereof.

18. The dental material of claim 11 wherein the polymeric matrix material further comprises polymerization initiators, polymerization accelerators, ultra-violet light absorbers, anti-oxidants, fluorescent whitening agents and mixtures thereof.

19. The dental material of claim 1 wherein the polymer matrices are visible light curing, self-curing, dual curing, and vacuum-, heat, and pressure-curable or a combination thereof.

20. The dental material of claim 19 wherein the visible light curable compositions comprise benzil diketones.

21. The dental material of claim 20 wherein the benzil diketones comprise dl-camphorquinone in an amount ranging from about 0.05 to 0.5 weight percent.

22. The dental material of claim 18 wherein the ultra-violet light absorbers comprise benzophenones, benzotriazoles, and mixtures thereof.

23. The dental material of claim 22 wherein the benzophenones comprise UV-9 and UV-5411.

24. The dental material of claim 22 wherein the benzotriazoles comprise 2-(2′-hydroxy-5′-methylphenyl) benzotriazole.

25. The dental material of claim 18 wherein the ultra-violet absorbers are present in an amount ranging from about 0.05 to about 5.0 weight percent.

26. The dental material of claim 18 wherein the polymerization accelerators comprise organic tertiary amines, acrylate derivatives, and mixtures thereof.

27. The dental material of claim 26 wherein the organic tertiary amines comprise dimethyl-p-toluidine, dihydroxyethyl-p-toluidine and mixtures thereof in an amount ranging from about 0.05 to about 4.0 weight percent.

28. The dental material of claim 26 wherein the acrylate derivatives comprise dimethylaminoethyl methacrylate in an amount in the range of from about 0.05 to 0.5 weight percent.

29. The dental material of claim 19 wherein the heat and pressure curable compositions comprise a heat cure initiator.

30. The dental material of claim 29 wherein the heat cure initiator comprises benzoyl peroxide, 1,1′-azobis(cyclohexanecarbonitrile), lauroyl peroxide, tributyl hydroperoxide, AIBN, benzoyl peroxide, 1,1′-azobis(cyclohexanecarbonitrile) or mixtures thereof.

31. The dental material of claim 1 wherein the additional filler materials are present in an amount in the range of about 5 to 95% by weight of the total dental material.

32. The dental material of claim 31 wherein the additional filler comprises powders, fibers or mixtures thereof.

33. The dental material of claim 31 wherein the filler comprises silica, silicate glass, quartz, barium silicate, strontium silicate, barium borosilicate, strontium borosilicate, borosilicate, lithium silicate, amorphous silica, ammoniated or deammoniated calcium phosphate and alumina, zirconia, tin oxide, titania, or mixtures thereof.

34. The dental material of claim 1 further comprising pigments, anti-oxidants, ultraviolet stabilizers, fluorescent whitening agents, dyes, medicaments and mixtures thereof.

35. The dental material of claim 34 wherein the anti-oxidants comprise BHT (2,6- di-tert-butyl-4-methylphenol), hydroquinone methyl ether or a mixture thereof in an amount in the range from about 0.1 to about 0.3% by weight.

36. The dental material of claim 34 wherein the ultraviolet stabilizers comprise benzophenones, benzotriazoles, and mixtures thereof.

37. The dental material of claim 36 wherein the benzophenones comprise 2-hydroxy-4- methoxybenzophenone.

38. The dental material of claim 36 wherein the benzotriazoles comprise 2-(2′-hydroxy-5′- methylphenyl) benzotriazole, 2-(2-hydroxy-5-tert- octylphenyl) benzotriazole or mixtures thereof.

39. The dental material of claim 34 wherein the fluorescent whitening agents comprise 2,5-bis(5-tert-butyl-2-benzoxazole) thiophene.

40. The dental material of claim 34 wherein the dyes comprise iron oxides, yellow No.5, yellow No. 6, or mixtures thereof.

41. The dental material of claim 34 wherein the medicaments comprise pain relieving agents, antibiotics, anti-inflammation agents, desensitizing agents, and mixtures thereof.

42. The dental material of claim 41 wherein the pain relieving agents comprise Novocaine (procaine hydrochloride), Benzocain (ethyl aminobenzoate), ascorbic acid, butacaine sulfonate, and dibutacaine hydrochloride, or mixtures thereof.

43. The dental material of claim 41 wherein the antibiotics comprise sulfadiazine, procaine penicillin, aureomycin, streptomycin, tetramycin, chloramphenicol, butabarbital, diethyl stilbestrol, or mixtures thereof.

44. The dental material of claim 41 wherein anti-inflammation agents comprise p-aminosalicylic acid, aspirin, chlorohexadine, or mixtures thereof.

45. The dental material of claim 41 wherein the desensitizing agents comprise sodium fluoride, potassium nitrate, or mixtures thereof.
